(19) United States
(12) Patent Application Publication
(10) Pub. No.: US 2006/0210603 A1
(43) Pub. Date: Sep. 21, 2006

(54) Implantable medical articles having laminin coatings and methods of use

(76) Inventors: Stuart K. Williams, Tucson, AZ (US); David E. Babcock, St. Louis Park, MN (US); Joseph A. Chinn, Shakopee, MN (US); David L. Clapper, Shorewood, MN (US)

Correspondence Address: WEAVER, PAUL;Kagan Binder PLLC, Maple Island Building, 221 Main St N Ste 200, Stillwater, MN, 55082, US

(21) Appl. No.: 11/360,284

(22) Filed: Feb. 23, 2006

Related U.S. Application Data
(60) Provisional application No. 60/655,576, filed on Feb. 23, 2005.

(51) Int. Cl. A61K 38/10; A61K 38/54; A61F 2/00

(52) U.S. Cl. 424/423; 514/13; 514/14

(57) ABSTRACT

Laminin-containing coatings for the surfaces of implantable medical devices are disclosed. The coatings promote the formation of vessels in association with the coated surfaces with minimal fibrotic resonse.

### CROSS-REFERENCE TO RELATED APPLICATIONS

The present non-provisional Application claims the benefit of provisional Application having Ser. No. 60/655,576, filed on Feb. 23, 2005, and entitled Surface Modification of Tubular Structures Supporting Differential Cellular Activity: Surface Modification of Polymers to Promote Neovascularization.

### FIELD OF THE INVENTION

The invention relates to methods for promoting a vascularizing response in association with an implantable medical article. In some aspects, the implantable medical article has a laminin-containing coating. In other aspects, the invention relates to implantable medical articles having a stably denucleated porous portion.

### BACKGROUND OF THE INVENTION

Until more recently, the primary focus of advances in implantable medical article technology has been to alter a structural characteristic of the article to improve its function within the body. However, it has become appreciated that function of the implanted device at the site of implantation can be greatly enhanced by improving the compatibility of the devices in the context of the tissue response that occurs as a result of the implantation. Ideally, improved compatibility would allow surfaces of the implanted device to mimic natural tissue exposed by an injury and provide an environment for the formation of normal tissue as a result of the healing process.

Despite being inert and nontoxic, implanted biomaterials associated with the device, such as various plastics and metals, often trigger foreign body reactions such as inflammation, fibrosis, infection, and thrombosis. If excessive, some of these reactions may cause the device to fail in vivo. A moderate cellular inflammatory response is commonly seen immediately following implantation, wherein leukocytes, activated macrophages, and foreign body giant cells are recruited to the surface of the implanted device. While the inflammatory response is common and generally a component of the healing process, it is often accompanied by the formation of a substantial fibrous matrix on the surface of the implanted device. Excessive fibrosis and fibrous matrix encapsulation is generally undesirable as this encapsulation can isolate the implanted device from the surrounding tissue, thereby hindering the vascularization of the implant.

The formation of new blood vessels, commonly seen as microvessels, is also a component of the tissue healing process. An angiogenic response refers to the formation of new blood vessels from pre-existing vessels. A vasculogenic response refers to the de novo formation of new blood vessels from single cells. The formation of new blood vessels is a complex process that is generally poorly understood, but appears to involve the recruitment of endothelial cells to the area of blood vessel formation.

Promoting an angiogenic response and the formation of new blood vessels in association with the implant surface is thought to improve the assimilation of the implant in the surrounding tissue environment. Improving the angiogenic response by modifying the properties of the implant may contribute to its long-term function by promoting the formation of new blood vessels which can allow for appropriate nutrient and waste product exchange to the surrounding tissues. An improved angiogenic response can be beneficial in other ways. For example, in the case of vascular grafts, an increase in vascular penetration through the interstitial thickness of a graft (neovascularization) could improve the patency of the vessel. Increased vascular penetration could provide a source of autologous endothelial cells for lumenal endothelialization, thereby forming a non-thrombogenic blood/tissue interface.

Improvements in biocompatibility leading to an increased angiogenic response can be objectively evaluated. For example, an angiogenic response can be quantitated by microscopically determining the microvessel density in association with the implant surface after a period of implantation. In addition to determining the extent of new microvessel growth, histology can be performed to determine the types of microvessel types that are formed in association with the surface of the implant. In this regard, both vascular growth and vascular complexity can be important factors in a healing response, and can be assessed following modifications to the surface of the device, and a period of implantation.

Furthermore, improvements in biocompatibility can also be measured by observing that the device elicits controlled inflammatory, and minimal fibrotic responses. Improvements in biocompatibility can also be measured by showing that these responses are different, or less than the magnitude of responses seen with other types of surface modifications.

Given this, modification of devices in a manner that mimics the natural healing of damaged tissues in the body, and which integrates the implanted article into normal tissue, has become realized as a way of greatly improving the functionality and functional life of the implanted device. Such modifications would ideally result in minimal or no fibrotic encapsulation and an increase in microvascular development in association with the implant surface. The modification of the surfaces of plastic or metal implantable medical devices with various natural and synthetic materials is commonly known in the art as a way of attempting to improve the biocompatibility of implantable devices.

One approach to improving the biocompatibility of implantable medicals articles involves modifying the implant to promote the migration of endothelial cells from adjacent tissue. Such modifications are thought to improve the formation of new blood vessels in association with the surface of the device. Attempts to provide a surface with improved biocompatibility have involved depositing extracellular matrix (ECM) proteins onto surfaces of implantable plastic devices. Stable formation of the proteins is desirable as it could promote the formation and persistence of new blood vessels.

The modification of ECM proteins with reactive groups has been shown as a way of improving the stability of the coatings. For example, fibronectin (FN) and collagen IV derivatized with photoreactive groups and immobilized on polyurethane (PU) and expanded polytetrafluoroethylene (ePTFE) vascular grafts enhance the in vitro attachment and growth of endothelial cells to the graft surfaces.

Furthermore, while the modification of device surfaces with certain extracellular matrix proteins may promote endothelial cells attachment, this attachment may not correlate with the capacity of the coated surfaces to promote angiogenesis. Further, such coated devices may also promote considerable inflammatory and fibrotic responses.

### SUMMARY

The present invention generally relates to implantable medical articles having coatings that improve the function of the article in vivo. The invention also relates to methods for using these coated-medical articles in a subject. In particular, the coatings of the present invention provide improved function of the article by promoting the formation of blood vessels in association with the coated surface.

In experimental studies associated with one aspect of the invention, it has been found that the immobilization of a laminin polypeptide on the surface of a medical implant significantly increased the formation of vascular growth associated with the coated surfaces of the article. In particular, a coating including laminin-5 was shown to cause the formation of blood vessels in association with the coated surface, as exemplified by the formation of microvessels throughout a porous ePTFE substrate having a laminin-5 coating. Notably, the formation of these microvessels occurred without the formation of a thick avascular fibrous capsule on the surface of the article and in the presence of a controlled inflammatory response.

Additional studies based on these finding revealed that the combination of a laminin, such as laminin-1 or laminin-5, and another adhesion factor, such as a collagen, also promoted excellent cell attachment and increased new vascular growth in association with surfaces that were coated with these materials.

Used in conjunction with an implantable medical article, the coatings of the present invention promote a wound-healing response that more closely mimics the natural wound-healing response of the body. This is indicated by the observed controlled inflammatory response, the minimal fibrotic response, and the formation of a dense network of microvessels associated with the coated surface of the implanted device.

This discovery provides an important improvement for the preparation and use of implantable medical devices. The substantial formation of blood vessels seen using the laminin-based coatings of the implant, in combination with the minimal fibrotic and controlled inflammatory responses, establishes parameters for improving the functionality of the implanted article, especially over an extended period of time. The coatings of the present invention provide an improvement over adhesion-factor coatings of the prior art, as the combination of these responses (i.e., new vascular growth, minimal inflammatory and fibrotic responses) in other coatings was not previously attainable.

In one aspect, the invention provides a method for causing the formation of blood vessels in association with a surface of an implantable medical article. The method can also be used when it is desired to minimize fibrotic responses associated with implantation of a medical article. The method includes a step of implanting a medical article having a coating in a subject. In one aspect, the coating includes laminin-5, an active portion thereof, or a binding member thereof, present in an amount sufficient to cause formation of blood vessels in association with a surface of the implanted medical article. Another step of the method involves maintaining the medical article in the subject for at least a period of time sufficient to cause formation of blood vessels in association with a surface of the implanted medical article.

In some aspects, the method is performed using a coating including laminin-5, an active portion thereof, or a binding member of thereof, is wherein the coating is formed by a method that includes a step of disposing a coating composition comprising laminin-5, an active portion thereof, or a binding member of thereof, at a concentration of 1 μm/mL or greater.

Additional studies revealed that the combination of a laminin and another adhesion factor also causes significant formation of blood vessels in association with the surface of the coated article and minimizes the fibrotic response. Therefore, the invention also provides a method that includes a step of implanting a medical article having a coating in a subject, the coating includes a first component comprising a laminin, an active portion thereof, or a binding member thereof, and a second component comprising an adhesion factor, an active portion thereof, or a binding member thereof.

One preferred coating includes laminin-5, an active portion thereof, or a binding member thereof, and collagen, an active portion thereof, or a binding member thereof. In some aspects the active portion of laminin-5 is the alpha 3 (α3) chain of laminin-5, the LG3 module of the (α3) chain, or the active peptide domains (such as PPFLMLLKGSTR and NSFMALYLSKGR) of the LG3 module.

Another preferred coating includes laminin-1, or an active portion thereof, or a binding member thereof and collagen, or an active portion thereof, or a binding member thereof. Preferred collagens are selected from the group of collagen I and collagen IV.

Generally, the coated article is maintained in the subject at least for a period of time sufficient for the formation vessels in association with the coated surface. For example, after four weeks of implantation, the microvessel density associated with the coated surface of the implant was greater than 100 vessels/cm². Furthermore, after this time period, minimal formation of a fibrous capsule was observed. The coatings of the present invention are particularly suitable for long-term implantable devices, such as those that reside in the body for a period of time of a month or longer.

In some cases, the step of implanting is performed by delivering the medical article to an intravascular location in the subject. For example, the article delivered intravascularly can be a selected form grafts, stents, stent-graft combinations, endografts, and shunts.

Preferably, the implantable medical article includes a porous portion. For example, the porous portion can include pores of a size sufficient to permit the in-growth or through-growth of vessels as promoted by the laminin-based coating. In some aspects of the invention, the porous portion can be formed from natural or synthetic materials, including polymeric materials formed into woven and/or non-woven fiber structures. In some aspects the porous structure includes ePTFE.

As exemplified by cylindrically-shaped intravascular grafts, the laminin-based coatings can promote the growth of new vessels from the ablumenal surface of the graft to the lumenal surface, without the formation of a thick cellular fibrotic capsule on either surface of the graft. In this regard, the laminin-based coating promotes the formation of a tissue-like structure including the porous graft portion that is highly vascularized and is able to exchange biological components such as nutrients and waste products, overall effectively integrating the implant within the surrounding tissue.

In some aspects of the invention, a coating that includes laminin-5 is formed on the surface of an implantable medical article by a method that comprises a step of (a) contacting the surface of the implantable medical article with a cell exudate enriched in laminin-5. Laminin-5, along with other polypeptide cofactors, may be deposited on the surface of the article to form the coating. For example, as one way of providing a laminin-containing coating to an article having a porous portion, a composition, such as a cell exudate, can be flowed through the article to force laminin, and any additional component, into the porous portion of the article, thereby depositing laminin on the surface of the porous portion. The method can include the steps of (a) providing a article having a porous portion (b) under pressure, flowing a composition comprising laminin through the porous portion, wherein laminin is deposited on the porous portion. Deposition of laminin on the surface can occur by adsorption.

Given the advantageous use of the present coating for promoting the formation of vessels in association with coated portion of an intravascular graft, the present invention also provides methods for the transmural endothelialization of an intravascular device comprising a porous portion. The method can include a step of maintaining the article comprising a laminin-based coating in a subject or a period of time sufficient to cause the growth of microvessels into the porous portion of the implantable device, and sufficient provide endothelial cells to the lumenal surface of the device via the microvessels.

It has also been discovered that enhanced coatings can be formed by combining a polypeptide comprising laminin, an active portion thereof, or a binding member thereof, with one or more other adhesion factors, an active portion thereof, or a binding member thereof, with one or more additional coating components. The one or more additional components can comprise a polymeric component, a first reactive group, and a second reactive group. The first reactive group allows for crosslinking of the polymeric component or the bonding of the polymeric component to the surface of the article, and the second reactive group allows for binding of laminin and the adhesion factors. Preferably, the polymeric component comprises a pendent first reactive group and a pendent second reactive group.

In some aspects, the first reactive group comprises a photoreactive group. The second reactive groups are individually reactive with laminin and the adhesion factor For example, second reactive groups can be amine-reactive groups individually bonding the amine bearing residues of laminin and the adhesion factor to the polymer.

The coating provides distinct advantages for the formation of coating having two or more polypeptide-based components (such as laminin and another adhesion factor). The coatings are easily formed and do not require the chemical modification of laminin and the other adhesion factor. For example, in a method for forming the coating, as one step in the coating process, the polymer component can be disposed on the surface of the article and treated to form a polymeric base layer, wherein the first reactive group covalently couples the polymer to the surface of the article, and/or the first reactive group covalently crosslinks the polymer to form a coated layer on the surface of the article. A subsequent step can involve disposing a composition including the laminin and the adhesion factor on the polymeric layer, wherein the laminin and the adhesion factor become individually bonded to the polymer component via second reactive groups. In this regard, processing steps are minimized. This improves efficiency and reduces costs associated with the coating procedure. In addition, laminin and another adhesion factor are stably presented on the device surface.

Therefore, in another aspect, the invention also provides an implantable medical article having a coating capable of causing the formation of vessels in association with a surface of the article. The coating includes a laminin, an active portion thereof, or a binding member thereof, and an adhesion factor, an active portion thereof, or a binding member thereof, the coating further comprising a polymeric component, a first group reacted to crosslink the polymeric component, and second groups reacted to individually bond the laminin and adhesion factor to the polymeric component.

In one aspect the coating includes laminin-5, or an active portion thereof, and collagen, preferably collagen I, or an active portion thereof, wherein the laminin-5 and collagen are independently bonded to the polymeric component via the second group, and the polymeric components are crosslinked via the first group. In another aspect the coating includes laminin-1 and collagen I.

In preparing the laminin-based coatings using a polymer component, it was advantageously discovered that the polymer base layer, in itself, provides a distinct advantage when used in association with an implantable article having a porous portion. It has been found that the polymer base layer, for instance, as provided using a polymer comprising a pendent first reactive group and a pendent second reactive group, allows the porous portion to remain stably denucleated during processing and use of the implantable article. Denucleation is a process of removing air bubbles trapped within interstices of certain porous materials, such as ePTFE. Denucleated ePTFE grafts have been shown to reduce the fibrous capsule previously associated with untreated ePTFE, in addition to increasing blood vessel development around and within the ePTFE (Boswell, C. A. and Williams, S. K., et al. J. Biomater. Sci Polymer Edn., 10:319-329) However, ePTFE can easily be renucleated during subsequent processing or handing, which can reduce graft effectiveness.

Accordingly, in another aspect, the invention provides an implantable medical article comprising a stably denucleated porous portion having a coating comprising a synthetic polymer. The implantable medical article comprising a stably denucleated porous portion can be formed by a method that includes the steps of (a) denucleating the porous portion; and (b) forming a layer comprising synthetic polymer on a surface of the porous portion. The stably denucleated medical article can be implanted in a subject with only the layer comprising the synthetic polymer, or one or more additional factors can be coupled to the layer comprising the synthetic polymer. For example, any of the laminin-based compositions can be coupled to the synthetic polymer as described herein.

In some preferred aspects, the polymer is a synthetic polymer comprising reactive groups, such as photoreactive groups. The synthetic polymer is also preferably hydrophilic. An exemplary synthetic polymer is a vinyl polymer, such as an acrylamide polymer.

### BRIEF DESCRIPTION OF THE DRAWINGS

FIG. 1a is a Western blot analysis showing the identification of the beta 3 chain of laminin-5 as identified in the protein collected from ePTFE post flow of HCM, indicating the deposition of laminin-5 onto the surface of ePTFE.

FIG. 1b is a Western blot analysis probing for of the presence of collagen I, collagen IV, fibronectin, laminin-1, and laminin-5 in HCM deposited protein on the ePTFE. Fibronectin, laminin-1, and laminin-5 were observed in the HCM deposited protein.

FIG. 1c is a Western blot analysis of the presence of the three chains of laminin-5 (the α3, β3, and γ2 chains) pre- and post-laminin-5 depletion column.

FIG. 2a is a graph of the number of HMVEC per HPF (high powered field) adhering to ePTFE unmodified or coated with HCM, laminin-5 depleted HCM, pure laminin-5, or DCS-PBS, and corresponding to FIGS. 2b-2f.

FIGS. 2b-2f are electron micrographs of the luminal surface of ePTFE tubes ePTFE unmodified or coated with HCM, laminin-5 depleted HCM, pure laminin-5, or DCS-PBS. The ePTFE unmodified or coated tubes were sodded with HMVEC to determine adhesion. FIGS. 2b-2f correspond to the results of graph 2a.

FIG. 3a is a graph of subcutaneous vascularization of ePTFE implants from mouse subcutaneous tissue, the implants unmodified or coated with HCM, laminin-5 depleted HCM, pure laminin-5, or DCS-PBS, as measured by the number of vessels per mm², and corresponding to FIGS. 3b-3f.

FIG. 3b-3f are light micrographs of GS-1 positive vessels associated with the cross sections of ePTFE implants from mouse subcutaneous tissue, the implants unmodified or coated with HCM, laminin-5 depleted HCM, pure laminin-5, or DCS-PBS, and corresponding to the results of graph 3a.

FIG. 4 is a graph of inflammatory response of ePTFE implants from mouse subcutaneous tissue, the implants unmodified or coated with HCM, laminin-5 depleted HCM, pure laminin-5, or DCS-PBS, as measured by the number of F4/80 positive cells associated with the implant (activated macrophages and monocytes) per mm².

FIG. 5a-5e are light micrographs of hematoxylin and eosin-stained tissue cross-sections containing ePTFE implants from mouse subcutaneous tissue, the implants unmodified or coated with HCM, laminin-5 depleted HCM, pure laminin-5, or DCS-PBS.

FIG. 6 is a histogram of the results of the reagent in combination with the five binary protein coatings.

FIG. 7 is a histogram of the results of the reagent alone and in combination with one binary coating.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

In one aspect, the present invention is based on findings relating to the ability of a laminin-based coating including to increase the formation of blood vessels in association with a surface of a coated implant. In particular, a conditioned cell medium that included laminin-5 was used to deposit secreted proteins onto the surface of ePTFE in a bioreactor system (see Example 1). The modified ePTFE substrates were tested for a vascular response (including angiogenesis and neovascularization), cell adhesion, inflammatory response, and fibrous capsule formation (see Examples 2-4).

Immunoblotting using antibodies against collagen I, collagen IV, fibronectin, laminin-1, and laminin-5 revealed that both fibronectin and laminin-1 were identified in addition to laminin-5 as proteins that were deposited from the surface of the conditioned media onto the ePTFE. While this group of proteins showed good cell adhesion of endothelial cells and vascularization of the ePTFE, and fibrous encapsulation of the implant was also seen. Selective depletion of the laminin-5 and coating of the ePTFE with laminin-5-delpleted conditioned media showed a significant reduction in the cell adhesion of endothelial cells and vascularization of the ePTFE, and a moderate reduction in the inflammatory response.

Based on these findings, purified laminin-5 was deposited onto ePTFE. While the coating with purified laminin showed good endothelial cell adhesion (although less than the cell adhesion observed using the coating derived from the conditioned media), the neovascularization of the ePTFE having the purified laminin-5 coating was surprisingly enhanced as compared to the coating derived from the conditioned media. In addition, the purified laminin-5 coated ePTFE demonstrated minimal tissue capsule thickness and a moderate inflammatory response.

Based on these findings, subsequent coatings were prepared to investigate the contribution of laminins, alone, or in combination with other adhesion factors, for cell adhesion and the generation of a neovascular response associated with the coated surface. In addition, coatings were also prepared using coupling components to improve formation of the coating containing the polypeptide based adhesion factors. A polymeric component comprising first and second reactive groups was used to improve the coating process and coating properties. In the process of forming the coatings, it was advantageously discovered that this polymer-based coating component allowed for the formation of an implantable medical article having a stably denucleated porous portion.

Particularly preferred coatings were found to include a combination of a laminin and a collagen. Exemplary combinations include laminin-5 and collagen I, and laminin-1 and collagen I.

The coatings, devices, and methods of the invention can be used for promoting the formation of blood vessels in association with the coated surface of the article. In some aspects the formation of vessels occurs in association with a porous surface. The formation of new blood vessels is shown by the angiogenic (the development of new vessels from preexisting vessels) or neovascularizing (formation of vessels within a porous portion of an implant) responses. In many aspects, the implantable medical article will have a complex geometry that can be innervated by new blood vessels, if conditions are suitable for the formation of these new vessels in the proximity of the coated surface, such as would be promoted by the laminin-based coatings of the present invention. Formation of blood vessels can allow the implant to function in agreement with the tissue surrounding the implant, as the vascularized implant more closely resembles natural tissue.

According to the invention, a laminin-based coating that causes formation of blood vessels in association with the coated of an implantable medical article is described. The implantable medical article can be an article that is introduced into a mammal for the prophylaxis or treatment of a medical condition.

Implantable medical articles include, but are not limited to vascular implants and grafts, grafts, surgical devices; synthetic prostheses; vascular prosthesis including stents, endoprosthesis, stent-graft, and endovascular-stent combinations; small diameter grafts, abdominal aortic aneurysm grafts; wound dressings and wound management devices; hemostatic barriers; mesh and hernia plugs; patches, including uterine bleeding patches, atrial septal defect (ASD) patches, patent foramen ovale (PFO) patches, ventricular septal defect (VSD) patches, pericardial patches, epicardial patches, and other generic cardiac patches; ASD, PFO, and VSD closures; percutaneous closure devices, mitral valve repair devices; heart valves, venous valves, aortic filters; venous filters; left atrial appendage filters; valve annuloplasty devices, catheters; neuroanuerysm patches; central venous access catheters, vascular access catheters, abscess drainage catheters, drug infusion catheters, parental feeding catheters, intravenous catheters (e.g., treated with antithrombotic agents), stroke therapy catheters, blood pressure and stent graft catheters; anastomosis devices and anastomotic closures; aneurysm exclusion devices; biosensors including glucose sensors; birth control devices; cosmetic implants including breast implants, lip implants, chin and cheek implants; cardiac sensors; infection control devices; membranes; tissue scaffolds; tissue-related materials including small intestinal submucosal (SIS) matrices; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices including bladder slings, renal devices and hemodialysis devices, colostomy bag attachment devices; biliary drainage products.

A medical article having a laminin-containing coating that causes formation of blood vessels in association with the coated surface can also be prepared by assembling an article having two or more “parts” (for example, pieces of a medical article that can be put together to form the article) wherein at least one of the parts has a coating. All or a portion of the part of the medical article can have a laminin-containing coating. In this regard, the invention also contemplates parts of medical articles (for example, not the fully assembled article) that have a laminin-containing coating.

The implantable medical article can be formed from any suitable material. General classes of materials from which the medical article can be formed include natural polymers, synthetic polymers, metals, and ceramics. Combinations of any of these general classes of materials can be used to form the implantable medical article.

Metals that can be used in the implantable medical articles include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35. The surface of an implantable metal article can be treated to facilitate formation of the laminin-containing coating. For example, an implantable medical article comprising a metal can include one or more base layers, such as a Parylene™ layer, or a silane-containing layer, such as hydroxy- or chloro-silane.

The implantable medical article can be formed from synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, dextran, dextran sulfate, polydimethylsiloxanes, and polyetherketone.

In one aspect of the invention, the medical article includes a halogenated polymer, such as a chlorinated and/or fluorinated polymers. For example, the laminin-containing coating can be formed on a surface of the implantable medical article that includes a perhalogenated polymer, such as a perfluorinated polymer

Examples of perhalogenated polymers that can be used as substrate materials include perfluoroalkoxy (PFA) polymers, such as Teflon™ and Neoflon™; polychlorotrifluoroethylene (PCTFE); fluorinated ethylene polymers (FEP), such as polymers of tetrafluoroethylene and hexafloropropylene; poly(tetrafluoroethylene) (PTFE); and ePTFE.

Other fluoropolymers are known in the art and described in various references, such as, W. Woebcken, Saechtling International Plastics Handbook for the Technologist, Engineer and User, 3^{rd} Ed., (Hanser Publishers, 1995) pp. 234-240.

In some aspects of the invention, the implantable medical article includes a porous portion and laminin-containing coating is formed on a surface of the porous portion. The porous portion can be constructed from one or a combination of similar or different biomaterials. The pores of the porous portion are preferably of a physical dimension that permits formation of vessels within the porous structure. For example, a suitable average pore size can be about 2 μm or greater, and preferably in the range of about 4 μm to about 150 μm.

In many cases the porous portion of the implantable medical article comprises a fiber or has fiber-like qualities. If the porous portion comprises a fiber it can be of any suitable diameter, ranging from fibers of nanometer diameters to millimeter diameters. Combinations of different sized fibers can also be present in the porous portion. The porous portion can be formed from a woven or non-woven material, or combinations thereof.

The porous surface can be formed from textiles, which include woven materials, knitted materials, and braided materials. Exemplary textile materials are woven materials that can be formed using any suitable weave pattern known in the art.

The porous surface can be that of a graft, sheath, cover, patch, sleeve, wrap, casing, and the like. These types of articles can function as the medical article itself or be used in conjunction with another part of a medical article.

The porous portion can optionally include stiffening materials to improve its the physical properties. For example, a stiffening material can improve the strength of a graft, thereby improving its patency.

In one exemplary aspect of the invention, the laminin-containing coating is formed on a porous PTFE substrate. The use of PTFE is well known in the art of implantable medical devices. PTFE tubes are commonly used as vascular grafts in the replacement or repair of a blood vessel. ePTFE tubes have a microporous structure consisting of small nodes interconnected with many tiny fibrilla. The spaces (i.e. pores) between the node surfaces that is spanned by the fibrils is defined as the internodal distance (IND). A graft having a large IND enhances tissue ingrowth and cell endothelization as the graft is inherently more porous. The porosity of an ePTFE vascular graft can be controlled by controlling the IND of the microporous structure of the tube.

Single or multi-layer ePTFE grafts can be used as substrates for the neovascularizing coatings. Examples of multi-layered ePTFE tubular structures useful as implantable prostheses are shown in U.S. Pat. Nos. 4,816,338; 4,478,898 and 5,001,276.

The laminin-containing coating can also be formed on other porous grafts, such as those that include velour-textured exteriors, with textured or smooth interiors. Grafts constructed from woven textile products are well known in the art and have been described in numerous documents, for example, U.S. Pat. No. 4,047,252; U.S. Pat. No. 5,178,630; U.S. Pat. No. 5,282,848; and U.S. Pat. No. 5,800,514.

Articles having porous portions also include stent-graft combinations.

As further example, another article that can include a laminin-containing coating is an aqueous drainage device, also called a seton or glaucoma shunt. These devices are used to relieve excess internal pressure of the eye (intra-ocular pressure; IOP) commonly associated with subjects suffering from glaucoma. The seton is positioned in tissue on the side of the eye and is connected to the inside portion of the front of the eye via a small tube. The tube allows drainage of the excess fluid from the eye, thereby lowering the IOP.

An aqueous drainage device comprising a porous portion, such as ePTFE, can be provided with a laminin-containing coating as described herein. The laminin-containing coating can increase the formation of vessels in the ePTFE, and reduce the formation of a fibrous capsule that is commonly associated with uncoated devices.

The implantable medical article can also be drug-eluting or drug-releasing. While the laminin and any other optional polypeptide components are generally coupled to the surface of the article, the article may also be capable of releasing a drug from a portion of the article. The drug-eluting or drug-releasing portion of the article can be on the same portion of the article that includes a laminin-based coating, or may be on a different portion of the article.

In some cases a hydrophilic drug, such as another polypeptide, that is not coupled to the surface of the device can be present in the coated layer that includes laminin. In these cases, the hydrophilic drug can be released from the coating while the laminin remains coupled to the surface.

In other cases the article includes a coated layer having a drug, wherein the drug is elutable or releasable from the coated layer. In preferred aspects this coated layer is a polymeric layer. For example, the coated layer that the drug is eluted or released from can included a polymer to which laminin is covalently bound. For example, a drug may be present in, and releasable from the coated layer that includes a polymer having a group that covalently binds laminin to the polymer.

The drug may also be present in a coated layer that includes a hydrophobic polymer. For example, the drug may be present in a coated layer that includes a poly(alkyl(meth)acrylate), such as polybutylmethacrylate (pBMA). The layer may also include other polymers, such as poly(ethylenevinylacetate) (pEVA); see U.S. Pat. No. 6,214,901. Other drug eluting polymer layers (such as those described in U.S. Pat. No. 6,669,980 poly(styrene-isobutylene-styrene); and U.S. Patent Publication Nos. 2005/0220843 and 2005/0244459) may be used.

Generally, the laminin-containing coating that is formed on the surface of the implantable medical article includes a laminin, or an active portion thereof. The laminin protein family includes multidomain glycoproteins that are naturally found in the basal lamina. Laminins are heterotrimers of three non-identical chains: one α, β, and γ chain that associate at the carboxy-termini into a coiled-coil structure to form a heterotrimeric molecule stabilized by disulfide linkages. Each laminin chain is a multidomain protein encoded by a distinct gene. Several isoforms of each chain have been described. Different alpha, beta, and gamma chain isoforms combine to give rise to different heterotrimeric laminin isoforms.

In one aspect of the invention, the coating on the implantable medical article includes laminin-5 or an active portion thereof. Laminin-5 is composed of the gamma 2 chain along with alpha 3 and beta 3 chains (laminin α3β3γ2) chains. It is synthesized initially as a 460 kD molecule that undergoes specific proteolytic cleavage to a smaller form after being secreted into the ECM. The size reduction is a result of processing the α3 and γ2 subunits from 190-200 to 160 kD and from 155 to 105 kD, respectively. Laminin-5 is an integral part of the anchoring filaments that connect epithelial cells to the underlying basement membrane.

The coating can include an active portion of laminin-5, which may be one or more of the chains of laminin-5, a portion of one of the chains, or combinations thereof, wherein the active portion is capable of causing the formation of blood vessels in association with the coated surface of the implant. In some aspects, the laminin α3 chain, or a portion thereof, is included in the coating on the implantable medical article. A portion of the laminin α3 chain has a globular structure and is referred to as the G domain, which, it itself, is composed of five tandem repeats referred to as LG repeats. One of the modules within the G domain, referred to as the LG3 module, has been shown to replicate key Ln-5 activities including cell adhesion, spreading, and migration (Shang, M., et al. (2001) J. Biol. Chem. 276:33045-33053. The sequence of the human LG3 modules is available as NCBI (National Center for Biotechnology Information) number A55347.

In one aspect the coating includes a polypeptide having the LG3 sequence of the laminin α3 chain.

Other shorter peptides within the G domain may also be used in the present coatings, such as the peptide sequences PPFLMLLKGSTR and NSFMALYLSKGR.

One advantage of using a portion of laminin-5 is that a higher density of laminin-5 activity may be able to be provided on the surface. Alternatively, less polypeptide may be required to provide the desired vascular response in association with the coating on the medical article.

Laminin-5 can be obtained from various cell lines including HaCaT (spontaneously immortalized human keratinocytes; Boukamp, P., et al. (1988) J. Cell Biol 106:761-771), and HT-1080 (human fibrosarcoma; ATCC, CCL-121). Polyclonal antibodies against laminin-5 are commercially available from, for example, Abcam (#ab14509; Cambridge, Mass.); monoclonal antibodies against laminin-5 chains are commercially available from, for example, Chemicon (mouse anti-laminin-5 γ2 subchain MAb; Temecula, Calif.) and Transduction Laboratories (mouse anti-laminin-5 β3 subchain MAb; Lexington, Ky.), or can be prepared based on a laminin-5 sequence (e.g., rabbit anti-laminin-5 a3 subchain polyclonal (RB-71) as prepared by Bethyl Laboratories, Inc. (Montgomery, Tex.) against the peptide CKANDITDEVLDGLNPIQTD (see Examples)).

Complete nucleic acid and protein sequences are available for the human laminin-5 α3, β3, and γ2 chains. Given this information and the techniques available to one of skill in the art, a desired laminin-5 portion, can be obtained using techniques such as immunopurification, recombinant protein products, or by peptide synthesis.

A coating having laminin-5 activity can also be prepared by providing a coating that includes a component that specifically binds to laminin-5, or a portion thereof, herein referred to as a “binding member.” Antibodies against laminin-5, and portions thereof, are commercially available and described herein. The coating can be prepared by substituting an antibody against laminin-5 for laminin-5 in the coating, or supplementing the coating with an antibody against laminin-5.

Laminin-5, a portion thereof, or a binding member thereof, can be coated on the surface of the implantable medical article in an amount sufficient to cause the formation of blood vessels in association with the coated surface. In some aspects laminin-5, or a portion thereof, is coated on the surface wherein the concentration of laminin-5 is about 1 μm/mL or greater in the coating composition.

In another aspect of the invention, laminin-5, or a portion thereof, is present as the predominant polypeptide in the coating. That is, laminin-5, or a portion thereof, is present at greater than 50% of the total amount of polypeptide present in the coating.

One or more other adhesion factor components can optionally be included in the coating. A coating that includes laminin-5 or an active portion thereof can also include another factor involved in cell adhesion. For example, the coating can include laminin-5 and another component selected from the group of factors that bind to a member of the integrin family of proteins. In one aspect the other component is be selected from the group of collagen, laminin-1, vitronectin, entactin, tenascin, thrombospondin, and ICAM, proteoglycans, elastin, hyaluronic acid, and active portions thereof. In some aspects fibronectin or fibrinogen can be included.

In some aspects, the coating includes a combination of laminin-5, or an active portion thereof, and a collagen, or an active portion thereof. For example, the coating can include a combination of laminin-5 and a collagen selected from collagen I and collagen IV. One exemplary combination includes a combination of laminin-5 and collagen I. In one mode of practice, laminin-5, or an active domain thereof, is present in the coating in an amount in the range of 50-99% of the total amount of polypeptide present in the coating, and collagen I is present in the coating in an amount in the range of 1-49% of the total amount of polypeptide present in the coating.

In another aspect of the invention, the coating includes laminin, such as laminin-1, or an active domain thereof, in combination with another factor involved in cell adhesion. For example, the coating can include laminin-1, or an active domain thereof, and another component selected from the group of factors that bind to a member of the integrin family of proteins, as described herein. For example, the coating can include a combination of laminin-1 and a factor selected from collagen, laminin-5, vitronectin, entactin, tenascin, thrombospondin, and ICAM (Intercellular Adhesion Molecule), and active portions thereof.

In some aspects, the coating can include a combination of laminin and a specific binding member or an antibody against a cell surface antigen involved in adhesion. For example, the coating can include laminin and an antibody against CD34, or a binding member of CD34, such as MadCAM or L-selectin. Anti-CD34 monoclonal antibodies can bind progenitor endothelial cells from human peripheral blood. These progenitor cells are capable of differentiating into endothelial cells. (Asahara et al. (1997) Science 275:964-967.) Hybridomas producing monoclonal antibodies directed against CD34 can be obtained from the American Type Tissue Collection. (Rockville, Md.).

The laminin-based coating can be formed in one or more ways. In some aspects, laminin, such as laminin-5 or laminin-1, or active domains thereof, and any additional component, are immobilized by deposition and adsorption onto the surface of the medical article. Typically, adsorption of polypeptide components is thought to be caused by non-covalent hydrophobic interactions between a portion of the polypeptide and the surface of the substrate. For the adsorption of polypeptides, the implantable medical article generally has a hydrophobic surface. The hydrophobic surface can be provided by the device material itself, such as halogenated thermoplastic such as ePTFE, or the surface of the device can be modified to provide a hydrophobic surface.

One or more polypeptide components can be immobilized on the surface by adsorption using any suitable method. If more than one component is immobilized, the process can be carried out wherein both of the components are immobilized simultaneously. For example, a mixture of laminin and collagen can be prepared and deposited on the surface of the article. Concentration of the components, the coating time, coating temperature, coating pH, ionic strength of the solution, presence of any additional reagents in the coating solution (such as detergents), can be chosen based on parameters know in the art to provide a suitable laminin-based coating on the surface of the article.

To exemplify one mode of immobilizing laminin by adhesion, coating of an ePTFE graft is described. Air is removed from the interstices of the ePTFE by treatment with an alcohol to provide a denucleated graft with decreased surface tension. For example, denucleation can be performed by successive submersions, starting with a solution with a high alcohol concentration (such as 100%) and decreasing the concentration of alcohol to a solution of deionized water. Alternatively, denucleation can be performed starting with an aqueous solution, changing to an alcohol solution. The graft can then be placed in PBS (for example, cation-free Phosphate Buffered Saline) prior to the coating process.

For the coating procedure, a coating composition that includes laminin is placed in contact with a surface of the ePTFE. In some modes of practice, for example, in the case of a tubular ePTFE substrate, the laminin composition can be pumped through the tubular portion for a predetermined period of time. In one mode of practice, the coating composition is placed in contact with the substrate for a period of time in the range of about 1 hour to about 12 hours.

Laminin can be present in the composition in an amount to provide a coating that can cause the formation of vessels in association with the coated surface. For example, laminin-5 can be present in the composition at a concentration of about 1 μg/mL or greater.

The composition can include laminin, such as laminin-5, in pure form, or laminin obtained from a source wherein laminin is enriched in the composition.

The amount of laminin deposited on the substrate can be determined by removing the deposited protein using a detergent, such as SDS, and then performing protein qualification using immunoblotting.

In some aspects of the invention, one or more components of the coating composition are immobilized on the surface of the device via a coating component. In some aspects, the coating component can be used to improve the stability of the components of the coating (for example, laminin and other optional components) on the surface of the device.

Generally, the polypeptide components (laminin or a combination of laminin and other polypeptide factors) of the coating can be immobilized by one of two different arrangements, or a combination of the two. In some aspects the coating component can be a coupling moiety. As one arrangement for improving the association of the components of the coating, the polypeptide components are associated with one another via the coupling moiety. In this arrangement, the components are crosslinked to one another to form a linked network of molecules on the surface of the article. For example, a plurality of laminin molecules can be crosslinked via the coupling moiety to form a coated layer of laminin molecules. Other components, such as second components, for example, selected from collagen, laminin-1, vitronectin, entactin, tenascin, thrombospondin, ICAM, active domains thereof, can be crosslinked with the laminin.

Crosslinking of the components deposited on the surface of the device can be caused by reacting a polypeptide component of the coating composition with a coupling moiety, wherein the device surface is generally non-reactive with the coupling moiety. For example, wherein the coupling moiety is a group activatable by thermal or light energy, and the resulting activated species reacts with components of the coating composition, but not the device surface, the coupling moiety reacts with a portion of the coating components (e.g., laminin) to form a network of covalently coupled polypeptides. The surface in contact with the coating composition is generally non-reactive with the coupling moiety, which is in some aspects is hydrophobic and a poor source of abstractable hydrogens. For example, the surface can be a fluoropolymer-containing surface such as ePTFE.

In some aspects of the invention, the coupling moiety comprises a photoreactive group. Photoreactive groups, broadly defined, are groups that respond to specific applied external light energy to undergo active specie generation with resultant covalent bonding to a target. Photoreactive groups are those groups of atoms in a molecule that retain their covalent bonds unchanged under conditions of storage but which, upon activation, form covalent bonds with other molecules. The photoreactive groups generate active species such as free radicals, nitrenes, carbenes, and excited states of ketones upon absorption of external electromagnetic or kinetic (thermal) energy. Photoreactive groups may be chosen to be responsive to various portions of the electromagnetic spectrum, and photoreactive groups that are responsive to ultraviolet, visible or infrared portions of the spectrum are preferred. Photoreactive groups, including those that are described herein, are well known in the art. The present invention contemplates the use of any suitable photoreactive group for formation of the inventive coatings as described herein.

Photoreactive groups can generate active species such as free radicals and particularly nitrenes, carbenes, and excited states of ketones, upon absorption of electromagnetic energy. Photoreactive groups can be chosen to be responsive to various portions of the electromagnetic spectrum. Those that are responsive to the ultraviolet and visible portions of the spectrum are typically used.

Photoreactive aryl ketones such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives can be used. Examples of aryl ketones include heterocyclic derivatives of anthrone, including acridone, xanthone, and thioxanthone, and their ring substituted derivatives. Some photoreactive groups include thioxanthone, and its derivatives, having excitation energies greater than about 360 nm.

These types of photoreactive groups, such as aryl ketones, are readily capable of undergoing the activation/inactivation/reactivation cycle described herein. Benzophenone is a particularly preferred latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—C—N₃) such as ethyl azidoformate and phenyl azidoformate; sulfonyl azides (—SO₂—N₃) such as benezensulfonyl azide; and phosphoryl azides [(RO)₂PON₃] such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetatoacetates (—CO—CN₂CO—O—) such as t-butyl alpha diazoacetoacetate.

Other photoreactive groups include the diazirines (—CHN₂) such as 3-trifluoromethyl-3-phenyldiazirine; and ketenes (CH═C═O) such as ketene and diphenylketene.

Referring to embodiments wherein the coating comprises a crosslinked layer of polypeptide components, the coating can be formed by providing a laminin comprising a photoreactive group (i.e., photo-laminin). In these aspects, photo-laminin can be activated to crosslink to other components in the coating composition, including other photo-laminins.

Alternatively, the coating can be formed by combining the components of the coating composition with a coupling moiety that is a photoreactive crosslinking agent. The photoactivatable crosslinking agent can be non-ionic or ionic. The photoactivatable cross-linking agent can include at least two latent photoreactive groups that can become chemically reactive when exposed to an appropriate actinic energy source.

For example, the laminin coating can be formed using a non-ionic photoactivatable cross-linking agent having the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”).

Ionic photoactivatable cross-linking agents can also be used to form the laminin coating. Some ionic photoactivatable cross-linking agents are compounds having the formula: X₁—Y—X₂, wherein Y is a radical containing at least one acidic group, basic group, or a salt of an acidic group or basic group. X₁ and X₂ are each independently a radical containing a latent photoreactive group. For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

As a preferred arrangement for improving the association of the polypeptide components of the coating, the polypeptide (including laminin) components are immobilized on the surface of the device using with one or more additional coating components. The one or more additional components can comprise a polymeric component, a first reactive group, and a second reactive group.

In some modes of practice, the first reactive group allows for crosslinking of polymeric components to form a coated layer. For example, the first reactive group can be activated to react and bond to another polymeric component, forming a network of polymeric components as a layer on the surface of the implantable medical article. Such a crosslinked network of polymeric components may be formed when there is little or no reactivity of the first reactive group and the surface of the article. In some cases, the first reactive group is pendent from the polymeric component. Preferably, the first reactive group includes a photo-reactive group as described herein.

Alternatively, the network of polymeric components formed as a layer on the surface of the implantable medical article is formed by the combining a polymeric component with a crosslinking agent, such as crosslinking agent comprising photoreactive groups, as described herein.

In some cases, the polymeric component is coupled to the surface of the article by the reaction of the first reactive group, such a photoreactive group, with the surface of the article. In this case, the polymeric component can be covalently bonded to the surface of the article.

The second reactive group allows for bonding of laminin and in some cases, other adhesion factors. The second reactive groups are individually reactive with laminin and the adhesion factor. For example, second reactive groups can be amine-reactive groups, such as N-oxysuccinimide (NOS) groups. Other amine-reactive groups include, aldehyde, isothiocyanate, bromoacetyl, chloroacetyl, iodoacetyl, anhydride, isocyanate and maleimide groups.

The second reactive group can also be pendent from the polymeric component. Preferably, the polymeric component comprises a pendent first reactive group and a pendent second reactive group. Use of a polymeric component with pendent first and second reactive groups provides distinct processing and functional advantages. For example, the polymeric component with these pendent groups can be disposed on a surface of the article, and treated to activate the first reactive group to form a coated layer. Subsequently, laminin can be disposed on the surface to react with the second reactive group, effectively immobilizing laminin on the surface.

This arrangement is particularly advantageous when a combination of laminin and another adhesion factor are immobilized on the surface, such as a combination of laminin-5 and collagen. Prior to disposing, these polypeptide components (including laminin) can be combined at a desired ratio or concentrations, and then disposed on the polymeric component with reactive second groups. Each polypeptide component can individually react with second reactive groups coupling the polypeptides to the polymer component. In this regard, processing steps are minimized. These improve the efficiency and reduce costs associated with the coating procedure.

In a preferred aspect, the polymer (coating component) comprises a hydrophilic polymer. The hydrophilic polymer that is used to form the laminin-containing coating can be a synthetic polymer, a natural polymer, or a derivative of a natural polymer. Exemplary natural hydrophilic polymers include carboxymethylcellulose, hydroxymethylcellulose, derivatives of these polymers, and similar natural hydrophilic polymers and derivatives thereof.

In another preferred aspect, the polymer is hydrophilic and synthetic. Synthetic hydrophilic polymers can be prepared from any suitable monomer including acrylic monomers, vinyl monomers, ether monomers, or combinations of any one or more of these types of monomers. Acrylic monomers include, for example, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, and derivatives and/or mixtures of any of these. Vinyl monomers include, for example, vinyl acetate, vinylpyrrolidone, vinyl alcohol, and derivatives of any of these. Ether monomers include, for example, ethylene oxide, propylene oxide, butylene oxide, and derivatives of any of these. Examples of polymers that can be formed from these monomers include poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, for example, methyl vinyl ether/maleic anhydride copolymers and vinyl pyrrolidone/(meth)acrylamide copolymers. Mixtures of homopolymers and/or copolymers can be used.

In exemplary modes of practice the hydrophilic polymer is a (meth)acrylamide copolymer, such as one formed from (meth)acrylamide and (meth)acrylamide derivatives.

Use of a polymer-based coating component provides distinct processing, fuictional, and economic advantages in the preparation of a coating on an implantable medical article. For example, in a method for forming the coating, as one step in the coating process, the polymer coating component can be disposed on the surface of the article and treated to form a polymeric base layer, wherein the first reactive group is activated to covalently couple the polymer to the surface of the article, and/or the first reactive group covalently crosslinks the polymer to form a coated layer. A subsequent step can involve disposing a composition including one or more polypeptide components (laminin or a combination of laminin and other polypeptide factors) on the polymeric layer, wherein the first and second components become bonded to the polymer via second reactive groups.

In the course of preparing the coating using the polymeric coating component, it was found that use of the polymeric component to form a coated layer prior to disposing laminin resulted in additional processing and functional advantages.

In providing a coating to an ePTFE graft, steps were performed to denucleate the pores of the ePTFE, referring to the process of removing air bubbles from the pores. Generally, denucleation can be performed by treating the ePTFE with an primarily alcohol-based solution(s) and then subsequently transferring to a primarily aqueous solution, such as PBS. This process is generally beneficial as it increases the surface area that can be contacted by body fluids and tissue components following implantation of the graft, resulting in reduced fibrous capsule formation and increased blood vessel development around and within the ePTFE (Boswell, C. A. and Williams, S. K., et al. J Biomater. Sci Polymer Edn., 10:319-329).

However, ePTFE can easily be renucleated (air bubbles can be reintroduced into the porous portion), displacing the aqueous solution, during subsequent processing or handing. Generally, renucleation of ePTFE grafts can be observed as a change in the appearance of the material. Other techniques can be used to determine relative denucleation or renucleation. Renucleation can reduce graft effectiveness.

It was discovered that following the step of providing a base layer of polymeric material during the coating process, the ePTFE graft was able to remain “stably denucleated.” In a stably denucleated porous portion (such as a stable denucleated ePTFE graft), it is difficult to reintroduce air bubbles into the porous portion. That is, the aqueous solution is not readily displaced by small air pockets.

An implantable medical article having a stably denucleated porous portion can provide distinct processing and functional advantages. For example, an implantable medical article with a stably denucleated porous portion can be subject to handling steps that would otherwise renucleate the porous portion of the article. In this regard, processing steps that may be used to keep a porous article denucleated, such as specific storage or handling steps, may not be required.

An implantable medical article having a stable denucleated porous portion can be subsequently coated with a desired composition. The composition can be any laminin-containing compositions as described herein. Alternatively, other types of biomolecules can be coated on the stably denucleated portion as described herein.

The invention will be further described with reference to the following non-limiting Examples.

### Testing and Analysis

Western Blot

Deposition of laminin-5 onto ePTFE at the four time points of conditioned medium flow in the bioreactor system, and the conditioned medium samples pre- and post-flow over the antibody BM165 (University of Arizona; Dr. Stuart K. Williams) immunoaffinity column were evaluated by Western Blot analysis. Protein deposited onto the ePTFE was collected by gently agitating the ePTFE samples while they soaked in 500 μL of Laemmli SDS sample buffer and 10% 2-β-mercaptoethanol at 37° C. for 24 hrs. Conditioned medium samples were concentrated using Centricon YM30 (Centricon Centrifugal Filter Devices, Millipore Co., Bedford, Mass.) according to the manufactures guidelines. Protein concentration was determined using a Micro BCA kit (Pierce, Rockford, Ill.).

7% sodium dodecyl sulfate polyacrylamide gel electrophoresis (SDS-PAGE) was performed using 20 μl of each protein sample from the bioreactor modification or the volume equal to 20 μg of protein for the conditioned medium samples. The gel was then transferred to a polyvinylidene fluoride membrane (PVDF), Immobilon-P (Millipore Corp., Bedford, Mass.). Blots were stained with Ponceau S and when necessary, cut into individual strips for analysis.

Proteins were detected using specific antibodies; (1) rabbit anti-collagen I polyclonal (COL1.1; abcam, UK) 1:7500, 2) mouse anti-collagen IV monoclonal (catalog # MAB1910; Chemicon, Temecula, Calif.) 1:10,000, 3) mouse anti-fibronectin monoclonal (clone FN-15; Sigma, St. Louis, Mo.) 1:10,000, 4) rabbit anti-laminin-1 polyclonal (product # L-9393; Sigma, St. Louis, Mo.) 1:7500, 5) mouse anti-laminin-5 P3 subchain monoclonal (clone 17; Transduction Laboratories, Lexington, Ky.) 1:1500, 6) mouse anti-laminin-5 γ2 subchain monoclonal (catalog # MAB19562; Chemicon, Temecula, Calif.) 1:5000, 7) rabbit anti-laminin-5 α3 subchain polyclonal (RB-71, custom made by Bethyl Laboratories, Inc. against the peptide sequence CKANDITDEVLDGLNPIQTD originally identified by Champliaud et al. (Champliaud, M. F. et al. Human amnion contains a novel laminin variant, laminin 7, which like laminin 6, covalently associates with laminin 5 to promote stable epithelial-stromal attachment. J Cell Biol 132, 1189-1198 (1996), 1:5000 and observed using SuperSignal™ Substrate according to manufacturer's instructions (Pierce, Rockford, Ill.). Two secondary antibodies conjugated to horseradish peroxidase, rat anti-mouse IgG (clone LO-MG1-2; Serotec, Raleigh, N.C.) 1:5000, and goat anti-rabbit IgG (product # A9169; Sigma, St. Louis, Mo.) 1:5000, were used. Protein standards consisted of human collagen I, collagen IV, fibronectin, EHS laminin-1 (all from Becton Dickinson, San Jose, Calif.), and purified laminin-5.

Cell Adhesion to ePTFE

Confluent monolayers of human microvessel endothelial cells (HMVECs) were prepared for adhesion studies by treatment with 5 mM ethylene diamine tetraacetic acid (EDTA) in Dulbeccos Modified Eagle Media (DMEM) at 37° C. for 20 min. Suspended cells were collected into serum free medium (M199) containing 0.1% bovine serum albumin (BSA), 2 mM L-glutamine, and 5 mM HEPES buffer. The cells were sodded at a density of 2×10⁵ cells/cm² as described previously with minor changes by Williams, S. K et al. (Williams, S. K., Schneider, T., Kapelan, B. & Jarrell, B. E. Formation of a Functional Endothelium on Vascular Grafts. J Electron Microsc Tech 19, 439-451 (1991)). Briefly, cells were pressure sodded onto the lumenal surface of each ePTFE tube and allowed to adhere for 1 hour while rotating in an incubator at 37° C. and 5% CO₂. Following this incubation period, ePTFE samples were collected and placed in a formalin fixative.

Quantification of HMVEC Adhesion to ePTFE

Adherent cells were labeled with the DNA intercalater, Bisbenzimide (BBI), which fluoresces under UV light. Each sample was visualized using epi-fluorescence under a 10× objective using an UV filter. Five fields were randomly selected, images were captured into a computer based morphmetric system (Metamorph Imaging Systems Software; Universal Imaging Corporation, West Chester, Pa.), and cellular density was calculated based.

Scanning Electron Microscopy

Samples were prepared for scanning electron microscopy evaluation by dehydration, critical point drying, and sputter coating using a gold target. The samples were evaluated and photomicrographs obtained using a JEOL 820 scanning electron microscope (JEOL USA, Peabody, Mass.).

Implant Study Design

All animal studies were performed with protocols approved by the University of Arizona IACUC and according to the National Institutes of Health Guidelines for the Care and Use of Laboratory Animals (#85-23 Rev. 1985). Studies were limited to the subcutaneous tissue of mice. Surgeries were performed as previously described by Salzmann, D. L et al. (Salzmann, D. L., Kleinert, L. B., Berman, S. S. & Williams, S. K. The effects of porosity on endothelialization of ePTFE implanted in subcutaneous and adipose tissue. J. Biomed Mater Res 34, 463-476 (1997)).

Fibrous Encapsulation Evaluation

An evaluation of the tissue capsule that develops surrounding implants was performed on the first series of implants (HCM series). Five random images were captured at either the lumenal or ablumenal edge of the polymer from each haematoxylin and Eosin (H&E) stained section using a 20× objective and a Sony catseye camera. These images were categorized based on their position relative to the ePTFE disc (lumenal or ablumenal) as well as capsule tissue type (fibrous or cellular capsule). Using a computer based morphmetric system (Metamorph Imaging Systems Software; Universal Imaging Corporation, West Chester, Pa.), three measurements of the capsule thickness were taken from each image, totaling fifteen measurements per sample (five images per sample, three measurements per image). Values were expressed as mean thickness in μm±s.e.m.

Vessel Density

Vascular density was evaluated using the sections stained with Griffonia simplicifolia-1 (GS-1) (biotinylated lectin-GS-1; 1:250; Vector Laboratories, Burlingame, Calif.) viewed under a 40× water-immersion objective lens. The number of cross sectional and longitudinal vessel profiles were counted per high powered field (HPF) (HPF=54×54 μm²). The criterion for a positive vessel were, 1) positive GS-1 reaction, 2) an identifiable lumen, 3) located within the designated HPF area. These HPF were randomly selected at the tissue-polymer interface, along the entire outer curve of the implant disc, with 10 fields in the tissue and 10 fields in the ePTFE independently selected. Vascular density is expressed as mean number of vessels/mm²±s.e.m for each group.

Inflammation

Inflammatory response was evaluated using the sections stained with F4/80 viewed under a 40× water-immersion objective lens. Using a 54×54 μm² high power field, 10 fields were randomly selected in the tissue at the tissue-polymer interface, along the entire outer curve of the implant disc. F4/80 positively staining cells within the HPF were counted. Inflammatory response for each implant group was expressed as mean number of F4/80 positive cells/mm²±s.e.m.

Histology and Immunohistochemistry

Fixed tissue samples were dehydrated, embedded in paraffin, sectioned at 6 μm and processed for histological and immunocytochemical evaluation. General histological structure was determined with hematoxylin and eosin staining. The vasculature was identified using the lectin, GS-1. Samples were evaluated immunocytochemically for the presence of activated macrophages using an antibody against the F4/80 160 kD glycoprotein antigen (biotin-monoclonal, 1:100 Serotec, Inc., Raleigh, N.C.). A peroxidase conjugated streptavidin kit (Dako Inc., Carpinteria, Calif.) was used to detect binding for both evaluations, and samples were reacted with 3,3′ diaminobenzidine (DAB) substrate for visualization. Methyl green staining was used to identify background nuclei following both immunocytochemical techniques.

### EXAMPLE 1

In Vitro—Cell Culture

The HaCaT and II-4 cell lines (Dr. Norbert Fusenig (German Cancer Research Center) were maintained in culture medium (Dulbecco's Modified Eagle's Medium with high glucose, 10% fetal bovine serum, 2 mM L-glutamine, and 5 mM HEPES buffer). Cells at 70% confluence were rinsed with di-cation free phosphate buffered saline (DCF-PBS), pH 7.4, and placed in serum free medium for 48 hrs prior to collection of conditioned medium. Collected conditioned medium was centrifuged at 750 g for 5 min to remove debris prior to coating procedure.

Human microvessel endothelial cells (HMVEC) were isolated from human liposuction fat as previously described in Williams et al. (Williams, S. K., Wang, T. F., Castrillo, R. & Jarrell, B. E. Liposuction-derived human fat used for vascular graft sodding contains endothelial cells and not mesothelial cells as the major cell type. J Vasc Surg 19, 916-923 (1994)). Cells were maintained in culture medium (Medium 199, 10% fetal bovine serum, 60 μg/ml crude endothelial cell growth factor (ECGS), 2 mM L-glutamine, and 5 mM HEPES buffer) and used between passage-2 and passage-5.

Purification/removal of Laminin-5 from the Conditioned Medium

Laminin-5 purification was performed according to the procedure of Champliaud et al. (Champliaud, M. F. et al. Human amnion contains a novel laminin variant, laminin 7, which like laminin 6, covalently associates with laminin 5 to promote stable epithelial-stromal attachment. J Cell Biol 132, 1189-1198 (1996)) with minor variations. Briefly, differences from this method included the source of laminin-5; laminin-5 was obtained from the cell culture supernatant of HaCaT cells rather than from human amnion. Additionally, immunoaffinity chromatography using a Sepharose column complexed with monoclonal anti-laminin antibody, BM165 targeted at the α3 chain of laminin-5 was used.

Removal of laminin-5 from conditioned medium (in order to prepare HaCaT conditioned media-Ln5) was performed the same day as the adhesion experiment. Sepharose beads complexed with the monoclonal anti-laminin α3 chain antibody, BM165. A column was prepared using 300 μl of the conjugated beads. Conditioned medium was passed over the column a total of two times. The beads were regenerated in between passes using 1M acetic acid and rinsing with Dulbecco's cation-free phosphate-buffered saline (DCF-PBS) to remove the acid. Pre and post column samples were collected for western blot analysis and confirmation of laminin-5 removal.

Surface Modification

In preparation for modification of ePTFE (4 mm diameter tubular graft material, IMPRA, Inc., Tempe, Ariz.) with conditioned medium, the air was removed from the interstices of the material using successive ethanol submersions starting at 100% and decreasing by 10% increments to deionized water over 20 min. intervals. This process is referred to as denucleation, and results in the removal of air and the production of a graft with decreased surface tension. Following denucleation, ePTFE was placed in DCF-PBS for 1 hour prior to the bioreactor procedure.

For the coating procedure, tubular ePTFE, with the distal end capped, was placed in a bioreactor as described in US provisional application U.S. 60/655,576, filed Feb. 23, 2005. Approximately, 55 mls of HaCaT conditioned medium (HCM) was pumped through the tubular ePTFE at 15 ml/min. for either 1, 3, 6, or 12 hours. One hour flow regimens were used for the HCM and HCM minus laminin-5 groups (HCM-Ln5). DCF-PBS and purified laminin-5 modifications were also evaluated. Following denucleation, the DCF-PBS group was soaked in DCF-PBS over night and the pure laminin-5 group (1 ug/cm²) was coated and kept in DCF-PBS/laminin-5 solution at 4° C. overnight prior to cellular attachment studies. Additionally, samples were treated with EDTA to determine if calcium was required for laminin-5 deposition onto ePTFE. Samples were placed in a 4 mM EDTA bath post-modification for 24 h with gentle agitation prior to protein collection.

Western Blot Analysis

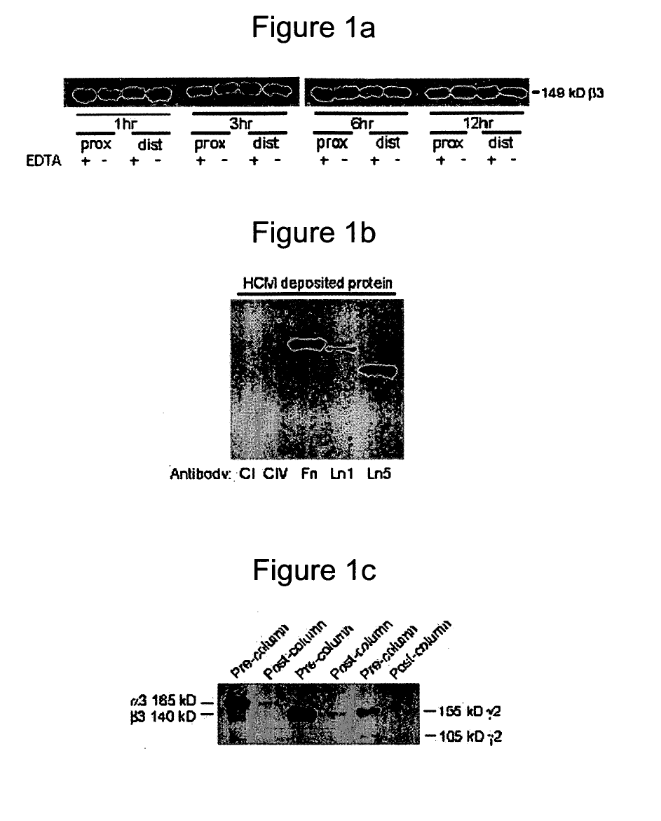

In FIG. 1(a) The beta 3 chain of laminin-5 was identified in the protein collected from ePTFE post-flow of HCM, confirming the deposition of laminin-5 onto the surface of ePTFE. Lanes are sorted by duration of flow (1, 3, 6, or 12 hrs). In FIG. 1(b) Multiple extracellular matrix proteins were identified in the protein deposited by the HCM onto ePTFE. Protein standards consisted of collagen I (CI), collagen IV (CIV), fibronectin (FN), laminin 1 (Ln1) and HaCaT cell lysate (Ln5). FN, Ln1, and Ln5 (β3 chain) were observed in the HCM deposited protein. In FIG. 1(c) Laminin-5 was successfully removed from the HCM. Each of the three chains of laminin-5, α3, β3, and γ2 were probed for. Minimal amounts of the α3 and β3 chains remained while the γ2 was completely removed.

Cell Adhesion to ePTFE

Confluent monolayers of human microvessel endothelial cells (HMVECs) were prepared for adhesion studies by treatment with 5 mM EDTA in DMEM at 37° C. for 20 min. Suspended cells were collected into serum free medium (M199) containing 0.1% BSA, 2 mM L-glutamine, and 5 mM HEPES buffer. The cells were sodded at a density of 2×10⁵ cells/cm² as described previously with minor changes by Williams, S. K et al. (Williams, S. K., Schneider, T., Kapelan, B. & Jarrell, B. E. Formation of a Functional Endothelium on Vascular Grafts. J Electron Microsc Tech 19, 439-451 (1991)). Briefly, cells were pressure sodded onto the lumenal surface of each ePTFE tube and allowed to adhere for 1 hour while rotating in an incubator at 37° C. and 5% CO₂. Following this incubation period, ePTFE samples were collected and placed in formalin fixative.

Quantification of HMVEC Adhesion to ePTFE

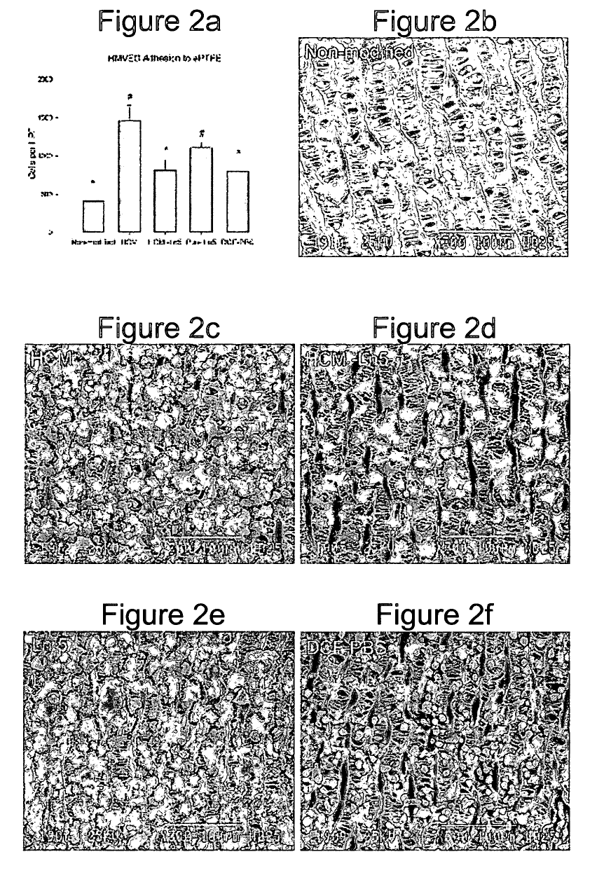

The histogram, FIG. 2a, shows the results of quantifying the HMVEC adhesion to modified ePTFE. Values expressed as mean number of cells per HPF. Both the HCM and pure laminin-5 modifications resulted in an increase in adhesion compared to non-modified ePTFE. FIGS. 2b-2f are scanning electron micrograph of the lumenal surface of the ePTFE tubes sodded with human microvessel endothelial cells (HMVEC). EPTFE modifications include non-modified, HaCaT conditioned medium (HCM), HCM minus laminin-5, pure laminin-5, and DCF-PBS modified ePTFE. The bar equals 100 μm. HMVEC are rounded on the DCF-PBS and non-modified samples, while they are spread on the conditioned medium and laminin-5 modified surfaces. The scanning electron micrographs visually reflect the results seen in the histogram of FIG. 2a.

Scanning Electron Microscopy

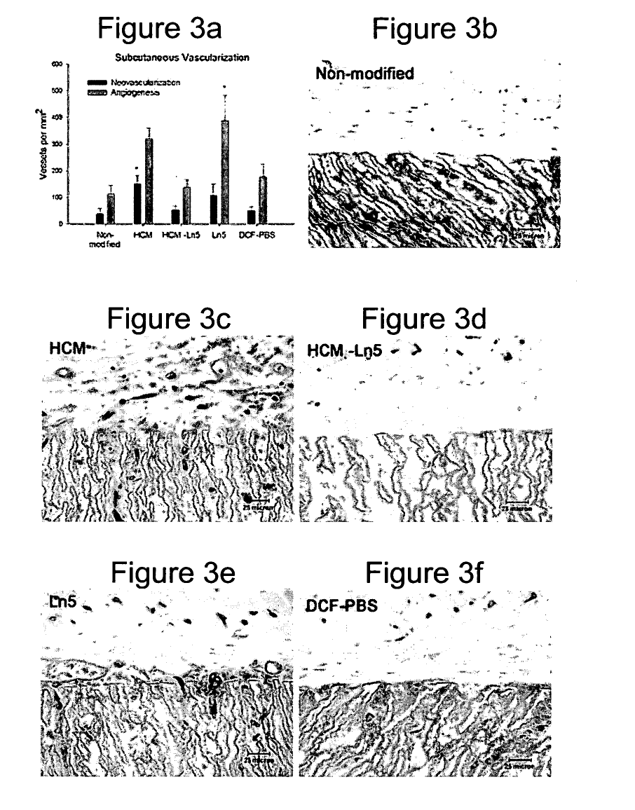

In FIG. 3a, the histogram shows the results of quantifying the angiogenic and neovascular response associated with modified and non-modified ePTFE implanted in mouse subcutaneous tissue. Values expressed as mean number of vessels per mm². HCM-Ln5, and DCF-PBS groups showed activity for the angiogenesis evaluation, Neovascularization is shown for HCM groups. FIGS. 3b-3f are light micrographs of GS-1 positive vessels associated with the cross sections of ePTFE implants from mouse subcutaneous tissue, the implants unmodified or coated with HCM, laminin-5 depleted HCM, pure laminin-5, or DCS-PBS, and corresponding to the results of FIG. 3a.

Implant Study Design

For each procedure, the animals were anesthetized with an intraperitoneal injection of 400 mg/kg avertin prior to the surgery. ePTFE discs (punches prepared from 4 mm diameter tubular graft material using a 4 mm biopsy punch) were implanted into the right and left rear haunch subcutaneous tissue in a random order with a total of two samples per animal (n=4/group). Samples were removed after the five week implant duration and placed in HistochoiceTm fixative (Amresco, Solon, Ohio). Samples consisted of ePTFE modified with HaCaT conditioned medium (HCM), HCM minus Laminin-5, Laminin-5, DCF-PBS or denucleated, and non-modified ePTFE implanted in a random order with a total of four samples per animal (n=4/group). Post modification, ePTFE discs were implanted subcutaneously in a total of fifteen, male 129-SVJ mice.

Fibrous Encapsulation Evaluation

An evaluation of the tissue capsule that develops surrounding implants was performed on the first series of implants (HCM series). Five random images were captured at either the lumenal or ablumenal edge of the polymer from each H&E stained section using a 20× objective and a Sony catseye camera. Using a computer based morphmetric system, these images were categorized based on their position relative to the ePTFE disc (lumenal or ablumenal) as well as capsule tissue type (fibrous or cellular capsule). Laminin 5 produced measurable ablumenal, lumenal and cellular effects.

TABLE 1

Subcutaneous

| | | Thickness | |
|---|---|---|---|
| | Surface | (micron) | % Cellular |
| HCM | Ablumenal | 58.6 ± 5 | 6 |
| | Lumenal | 106 ± 9 | 44 |
| HCM - | Ablumenal | 58.7 ± 5 | 12 |
| Laminin-5 | Lumenal | 89 ± 10 | 34 |
| Laminin-5 | Ablumenal | 46 ± 4 | 0 |
| | Lumenal | 50 ± 7 | 6 |
| DCF-PBS | Ablumenal | 45 ± 3 | 0 |
| | Lumenal | 82 ± 19 | 28 |
| Non-modified | Ablumenal | 61 ± 6 | 0 |
| | Lumenal | 81 ± 15 | 24 |

Inflammation Response

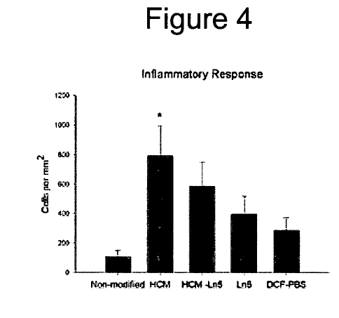

FIG. 4 is a graph of inflammatory response of F4/80 positive cells (activated macrophages and monocytes) associated with modified and non-modified ePTFE. F4/80 positive cells associated with ePTFE implanted in the mouse subcutaneous tissue. Values are expressed as mean number of cells per mm². No pattern is observed between the presence of laminin-5 in the modification and the extent of the inflammatory cell reaction.

Histology and Immunohistochemistry

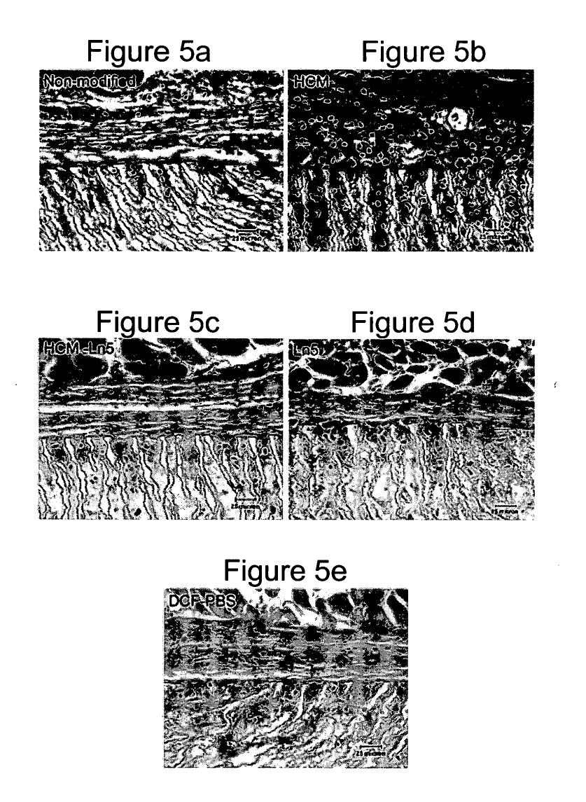

FIG. 5a-5b are light micrographs of hematoxylin and eosin-stained tissue cross-sections containing ePTFE implants from mouse subcutaneous tissue, the implants unmodified or coated with HCM, laminin-5 depleted HCM, pure laminin-5, or DCS-PBS. The bar equals 25 μm. An increased cellular response can be seen in association with the HCM modified sample, where as the laminin-5 modified sample has a thin, relatively acellular capsule formed around it.

### EXAMPLE 2

Binary Protein Coating Method

A heterobifunctional polyacrylamide reagent (HBPR, made as described in Example 9—U.S. Pat. No. 5,858,653) that contains amine-reactive and photo-reactive groups was used to immobilize extracellular matrix proteins onto ePTFE vascular graft (4 mm straight, C. R. Bard, Impra Corporation, Tempe, Ariz.). Matrix proteins were obtained from the following sources: bovine collagen-I (Kensey Nash), human collagen-IV (BD Biosciences), human fibronectin (BD Biosciences), mouse laminin-I (BD Biosciences), and human laminin-V (University of Arizona). Asceptic technique was used during all handling of the grafts and reagents. Grafts were cut to a 3.2 cm length. Female luer fittings (Small Parts, Inc.) were secured to each end of the graft with surgical suture. Grafts were denucleated (removing trapped air from the interstices of the graft) by soaking in isopropyl alcohol (IPA) for 20 minutes and then placing the graft in degassed Dulbecco's cation-free phosphate-buffered saline (DCF-PBS), pH 7.4. Grafts were removed from DCF-PBS, excess PBS was allowed to drip off, and the grafts were placed in a solution of HBPR (10 mg/ml in 50% IPA/water). After 30 minutes, the grafts were removed from the HBPR solution, dried (˜1.5 hours), and illuminated with a mercury arc flood lamp (emits strongly at 320-340 nm) for 3 minutes. The grafts were denucleated again as previously described. Matrix proteins were applied to the grafts from a single solution containing two different proteins in 0.1 M carbonate/bicarbonate (CBC) buffer, pH 9.0 (see Table 1). The distal end of the graft was capped and 12 ml of the protein solution was forced through the graft using a syringe and a 4-way male slip stopcock (Cole-Parmer). The HBPR-modified grafts were allowed to react with the proteins overnight at 4° C. The grafts were then rinsed briefly with DCF-PBS and evaluated for protein content and bioactivity (in vitro cell adhesion).

TABLE 2

| Binary Protein Coating | Coating Conc. |
|---|---|
| Solution | (ug/ml) |
| Collagen I/Fibronectin | 10/25 |
| Collagen I/Laminin V | 10/2.5 |
| Collagen I/Laminin I | 10/20 |
| Collagen IV/Laminin I | 5/20 |
| Laminin I/Fibronectin | 20/25 |

Immunofluorescence Staining Procedure

To confirm the presence of the proteins in the coatings an immunofluorescence staining procedure was employed. The following antibodies were used: rabbit anti-collagen-I (Rockland, Inc.), mouse anti-human collagen-IV (Chemicon), rabbit anti-mouse laminin-I (Sigma), mouse anti-human laminin-V (Transduction Laboratories), rabbit anti-human fibronectin (Sigma), goat anti-rabbit Texas Red (Rockland, Inc.), anti-mouse Alexa Fluor 350 (Molecular Probes), and goat anti-mouse Cy3 (Jackson Laboratories). Samples of graft were cut and placed in 12×75 mm plastic test tubes. Samples were then blocked with 2 ml 1.5% (w/v) BSA in tris-buffered saline (TBS) containing 0.05% Tween-20 for 20 minutes at room temperature on an orbital shaker. Next, samples were incubated with 0.4 ml primary antibody in DCF-PBS at room temperature for 1 hour on an orbital shaker. All grafts were then washed 3 times with 2 ml DCF-PBS, 15 minutes each, while shaking on an orbital shaker. Samples were incubated with 0.4 ml secondary antibody (fluorescent conjugate) in DCF-PBS at room temperature for 1 hour on an orbital shaker. Samples were then washed again with DCF-PBS as described previously. Luminal grafts were imaged with a fluorescence microscope using a 20× objective. All digital image parameters (contrast, brightness, etc.) were normalized to HBPR control.

Immunofluorescence Staining Results

Immunofluorescence staining with the HBPR reagent shows both collagen-I and laminin-I being detected on the binary protein coated graft. In other staining tests, collagen-I and laminin-V were detected. Similar results are seen for the other binary protein coatings (Table 3).

TABLE 3

| | HBPR/Protein | Fluorescence |
|---|---|---|
| | Coating | Prescence |
| Single Protein | COL IV | + |
| Coatings | COL I | + |
| | LM I | + |
| | FN | − |
| | LM V | + |
| Binary Protein | LM I/ | + |
| Coatings | FN | + |
| | COL IV/ | + |
| | LM I | + |
| | COL I/ | + |
| | FN | + |
| | COL I/ | + |
| | LM I | + |
| | COL I/ | + |
| | LM V | + |

Cell Adhesion Assay

Grafts were tested for acute cell adhesion to evaluate the bioactivity of each protein coating. Bovine aortic endothelial cells (BAECs) were dissociated and resuspended in culture media at 1×10⁶ cells/ml (passage 10 or less). A stopcock was attached to the proximal end of the test graft with the distal end open. With a syringe, 0.75 ml of well-mixed cell suspension was immediately delivered into the stopcock until a positive liquid meniscus was seen at the distal end. The stopcock was closed and the distal end was capped. Grafts were then placed in an incubator at 37° C. and 5% CO₂ for 30 minutes. The grafts were removed from the incubator and the luer fittings were cut off from both proximal and distal ends. A longitudinal cut was made with scissors to open the graft. Holding the end of graft with forceps, the graft was washed in DCF-PBS for about 5 seconds. The grafts were fixed in 8% paraformaldehyde in deionized water overnight at 4° C. Grafts were then stained with 4′,6-diamidino-2-phenylindole (DAPI, Sigma-Aldrich, Milwaukee, Wis.) and images were captured with a fluorescence microscope. Up to eight fields of view with the 20× objective were captured with each graft. Cell counts were determined and averaged.

Cell Adhesion Results

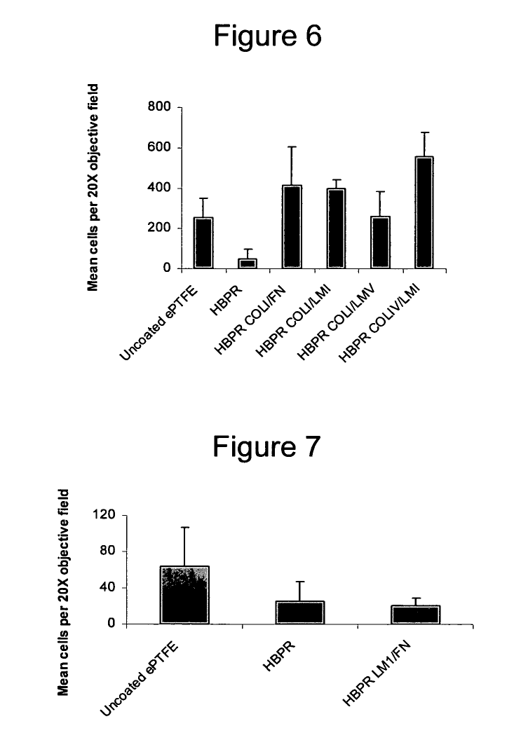

Four out of the five binary protein coatings enhanced cell adhesion 5 to 11-fold when compared to HBPR-only (FIG. 6). HBPR LMI/FN did not increase cell adhesion (FIG. 7).

### EXAMPLE 3

Rat Implant

An in vivo study evaluated the wound healing and inflammation associated with ePTFE discs coated with the reagent and protein coatings. ePTFE Discs (4 mm diameter size, (4 mm straight, C. R. Bard, Impra Corporation, Tempe, Ariz. A photoactivatable copolymer (HBPR) was prepared as described in Example 9 of U.S. Pat. No. 5,858,653. The following samples were evaluated: uncoated ePTFE, HBPR alone, HBPR Collagen-I, HBPR Laminin-I, HBPR Laminin-V, HBPR Collagen-I/Laminin-I, and HBPR Collagen-I/Laminin-V, Photo Collagen I and Photo Laminin 1. The laminin and collagen samples were obtained from the sources described in Example 2. Photo collagen 1 and Photo laminin 1 were made by the procedures described in Example 1 of U.S. Pat. No. 5,744,515, except that collagen 1 or laminin 1 was substituted were specifically made for this example. The coating procedure for HBPR and the protein samples is described in Example 2 except that the Collagen I/Laminin V example was prepared at 10/5.0 ug/ml. At the end of 4 weeks, the animals were anesthetized and the discs were excised and placed in Histochoice fixative. The animals were euthanized after material harvest using an overdose (100 mg/kg) of pentobarbital. The discs were sectioned, placed on slides and stained with H&E and immunohistochemically stained with GS-1. The ePTFE discs were explanted and processed for histology. Each disc was analyzed for peri-implant angiogenesis and neovascularization of the ePTFE graft material.

The treatments that most effectively support neovascularization of porous materials (in this case ePTFE) are HBPR Collagen-I/Laminin-I-V and the photolaminin 1. Photo collagen 1 and HBPR Collagen-I support surface angiogenesis but do not support extensive neovascularization. Uncoated ePTFE exhibits minimal angiogenesis and minimal neovascularization. The HBPR Laminin-V exhibited neovascularization greater than control but less than photo laminin 1.

### EXAMPLE 4

HBPR/protein-modified (HBPR COLI/LM5, etc) coronary stents (3×8 mm) are evaluated for healing responses in the iliac arteries of New Zealand white rabbits. The stents are crimped onto balloon catheters (3×15 mm) and are ethylene oxide sterilized. The stents are then deployed into New Zealand white rabbits, a test stent in one iliac artery and a bare metal stent control in the opposing artery. The stents are explanted at 7, 28 and 90 days and are evaluated by light and scanning electron microscopy. The explanted stents are cut in half longitudinally and are processed for histology. On one stent half, routine histopathological examination are performed from paraffin sections of the proximal and distal vessel up to the stent/vessel interface and plastic are embedded sections from the mid stent/vessel area. Appropriate stains hematoxylin and eosin (H&E), Masson's trichrome and elastic Van Gieson or equivalent are performed. Special emphasis is placed on endothelialization, neointimal thickness, inflammation, percent luminal stenosis, intimal fibrin content. To confirm the extent of endothelialization and thrombosis, the remaining half of each stent is processed for scanning electron microscopy.

What is claimed is:

1. A method for causing the formation of blood vessels in association with a coating on the surface of an implantable medical article comprising steps of:
implanting the medical article in a subject, the coating comprising laminin-5, an active portion thereof, or a binding member thereof; and
maintaining the medical article in the subject for a period of time sufficient to cause the formation of blood vessels in association with the coated surface.

2. The method of claim 1 wherein the step of implanting comprises delivering the medical article to an intravascular location in the subject.

3. The method of claim 1 wherein the step of maintaining results in the formation of a acellular fibrotic capsule of less than 100 mm in thickness on the implantable medical article.

4. The method of claim 1 wherein the step of maintaining results in the formation of a acellular fibrotic capsule of less than 75 mm in thickness on the implantable medical article.

5. A method for causing the formation of blood vessels in association with a coating on the surface of an implantable medical article comprising steps of:
implanting the medical article in a subject, the coating comprising
a laminin, an active portion thereof, or a binding member thereof, and
a collagen, an active portion thereof, or a binding member thereof,
maintaining the medical article in the subject for a period of time sufficient to cause the formation of blood vessels in association with the coated surface.

6. An implantable medical article having a coating comprising
a first component comprising laminin, an active portion thereof, or a binding member thereof, and
a second component comprising an adhesion factor, an active portion thereof, or a binding member thereof,
wherein the coating further comprises a polymeric component, a first group reacted to form a layer comprising the polymeric component, and second groups reacted to individually bond first and second components to the polymeric component.

7. The implantable medical article of claim 6 wherein the first component comprises laminin having a molecular weight of less than 500 kDa.

8. The implantable medical article of claim 6 wherein the first component comprises laminin-5.

9. The implantable medical article of claim 6 wherein the first component comprises the α3 chain of laminin-5.

10. The implantable medical article of claim 6 wherein the first component comprises the LG3 sequence of the α3 chain of laminin-5.

11. The implantable medical article of claim 6 wherein the first component comprises a laminin polypeptide sequence selected from PPFLMLLKGSTR, LAIKNDNLVYVY, DVISLYNFKHIY, TLFLAHGRLVFM, LVFMFNVGHKKL, and NSFMALYLSKGR.

12. The implantable medical article of claim 6 wherein the first component comprises proteinase-modified laminin-5.

13. The implantable medical article of claim 12 wherein the first component comprises metalloproteinase-modified laminin-5.

14. The implantable medical article of claim 6 wherein the first component comprises laminin-1

15. The implantable medical article of claim 6 wherein the second component comprises a collagen, an active portion thereof, or a binding member thereof.

16. The implantable medical article of claim 15 wherein the second component comprises a collagen I, an active portion thereof, or a binding member thereof.

17. The implantable medical article of claim 6 comprising a porous portion.

18. The implantable medical article of claim 17 wherein the porous portion is associated with a graft, sheath, or jacket.

19. The implantable medical article of claim 17 wherein the porous portion comprises a synthetic hydrophobic polymeric material.

20. The implantable medical article of claim 19 wherein the porous portion comprises ePTFE.

21. The implantable medical article of claim 6 wherein the polymeric component comprises a synthetic polymer.

22. The implantable medical article of claim 21 wherein the synthetic polymer is an acrylamide polymer.

23. The implantable medical article of claim 6 wherein the first group comprises a photoreactive group.

24. The implantable medical article of claim 6 wherein the second group comprises an amine-reactive group.

25. An implantable medical article comprising a stably denucleated porous portion.

26. The implantable medical article of claim 25 wherein the porous portion comprises a coated layer comprising a synthetic polymer.

27. The implantable medical article of claim 26 wherein the synthetic polymer comprises pendent photoreactive groups.

28. A method for preparing an implantable medical article comprising a stably denucleated porous portion comprising the steps of:
denucleating the porous portion; and
forming a coated layer on a surface of the porous portion, the coated layer comprising a synthetic polymer.
